SEQUENCE LISTING (23 sequences)

SEQ ID NO: 1 - protein, 277 aa, Homo sapiens
GALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 2 - protein, 98 aa, Homo sapiens
STNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEE

SEQ ID NO: 3 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 4 - protein, 98 aa, Homo sapiens; identical to SEQ ID NO: 2

SEQ ID NO: 5 - protein, 416 aa, Homo sapiens; identical to SEQ ID NO: 3

SEQ ID NO: 6 - protein, 416 aa, Homo sapiens; identical to SEQ ID NO: 3

SEQ ID NO: 7 - protein, 4 aa, Homo sapiens
ATPF

SEQ ID NO: 8 - protein, 278 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHL

SEQ ID NO: 9 - protein, 258 aa, Homo sapiens
IHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHL

SEQ ID NO: 10 - protein, 280 aa, Homo sapiens
AKPDRSSFVPSLFSKKKKNVTMRSIKTTRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGDRCKTLLEKPKLFFIQACRGTELDDGIQADSGPINDTDANPRYKIPVEADFLFAYSTVPGYYSWRSPGRGSWFVQALCSILEEHGKDLEIMQILTRVNDRVARHFESQSDDPHFHEKKQIPCVVSMLTKELYFSQ

SEQ ID NO: 11 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 12 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 13 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 14 - protein, 298 aa, Homo sapiens [sequence omitted]

SEQ ID NO: 15 - protein, 184 aa, Homo sapiens
AVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 16 - protein, 280 aa, Homo sapiens; identical to SEQ ID NO: 10

SEQ ID NO: 17 - protein, 117 aa, Homo sapiens
RDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSE

SEQ ID NO: 18 - protein, 124 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSR

SEQ ID NO: 19 - protein, 416 aa, Homo sapiens; identical to SEQ ID NO: 3

SEQ ID NO: 20 - protein, 98 aa, Homo sapiens; identical to SEQ ID NO: 2

SEQ ID NO: 21 - protein, 262 aa, Homo sapiens
GALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 22 - protein, 254 aa, Homo sapiens
GALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDSDIFVSYSTFPGFVSWRDPKSGSWYVETLDDIFEQWAHSEDLQSLLLRVANAVSVKGIYKQMPGCFNFLRKKLFFKTS

SEQ ID NO: 23 - protein, 277 aa, Homo sapiens
GALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCDNFLRKKLFFKTS

(19) United States
(12) Patent Application Publication
(10) Pub. No.: US 2009/0099826 A1
(43) Pub. Date: Apr. 16, 2009

(54) CASPASE-9:BIR3 DOMAIN OF XIAP COMPLEXES AND METHODS OF USE

(76) Inventor: Yigong Shi, Pennington, NJ (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/943,756

(22) Filed: Nov. 21, 2007

Related U.S. Application Data
(63) Continuation of application No. 10/769,218, filed on Jan. 30, 2004, now abandoned.
(60) Provisional application No. 60/443,950, filed on Jan. 31, 2003.

(51) Int. Cl. G06G 7/58; C07K 14/00

(52) U.S. Cl. 703/11; 530/350

(57) ABSTRACT

The present invention provides polypeptides and specific binding agents that modify the activity of an initiator caspase involved in apoptosis, caspase-9. The polypeptides include the third baculoviral IAP repeat (BIR3) of an IAP and form a heterodimer complex with caspase-9. Nucleic acid molecules including expression vectors encoding the polypeptides and variants thereof as well as variants of caspase-9 are provided. Such polypeptide and nucleic acid molecules may be used for modifying apoptosis.

### CROSS REFERENCE TO RELATED APPLICATIONS

This application claims the benefit of and priority to U.S. Non-Provisional Application No. 10/769,218 filed Jan. 30, 2004 which further claims priority to U.S. Provisional Application Ser. No. 60/443,590 filed Jan. 30, 2003, the contents of which are incorporated herein by reference in their entirety.

### GOVERNMENT INTERESTS

The United States Government may have certain rights to this invention pursuant to work funded by Grant No. CA90269.

### BACKGROUND AND SUMMARY

The inhibitor of apoptosis (IAP) family of proteins suppresses apoptosis by inhibiting the enzymatic activity of both the initiator and the effector caspases. At least eight members of the mammalian IAPs have been identified, including X-linked IAP (XIAP) (SEQ ID NO:13), c-IAP1 (SEQ ID NO:14), c-IAP2 (SEQ ID NO:15), and Livin/ML-IAP (SEQ ID NO:16). Each IAP protein contains 1-3 copies of the 80-residue zinc binding Baculoviral IAP Repeat (BIR). The different BIR domains and segments in the same IAP protein appear to exhibit distinct functions. For example, the third BIR domain (BIR3) of XIAP (SEQ ID NO:3) potentially inhibits the activity of the processed caspase-9 whereas the linker region between BIR1 and BIR2 selectively targets active caspases-3 or -7. The IAP-mediated inhibition of all caspases can be effectively removed by the mitochondrial protein Smac/DIABLO (SEQ ID NO:17), which is released into the cytoplasm during apoptosis. The pro-apoptotic activity of Smac/DIABLO depends on a four-amino-acid IAP-binding motif located at the N-terminus of the mature protein.

The mechanisms on the activation of the inhibition of the effector caspases have been well characterized in recent years. An active effector caspase, such as caspase-7, exists as a homo-dimer and contains two active sites, one from each monomer. Each active site is configured by four conserved surface loops (L1, L2, L3, and L4) from one monomer and a fifth supporting loop (L2′) from the adjacent monomer. The L2′ loop, which is indispensable for the formation of an active site, cannot adopt its productive conformation until after the activation cleavage. Hence the dimeric procaspase-7 zymogen (SEQ ID NO:18) is inactive because the L2′ loop exists in an unproductive (closed) conformation. The activation cleavage allows the L2′ loop to adopt the productive (open) conformation. The active site of caspase-3 or -7 can be tightly bound by a short peptide sequence in the linker region preceding the BIR2 domain of XIAP (SEQ ID NO:19). This binding occludes substrate entry and catalysis, resulting in the inhibition of caspases-3 or -7.

In contrast to the effector caspases, little is known about the activation and inhibition of the initiator caspases despite intense investigation. Extensive mutagenesis studies have identified several important residues in XIAP-BIR3 (SEQ ID NO:3) that are involved in the inhibition of the initiator caspase-9. In addition, an Smac-like tetrapeptide motif at the N-terminus of the small subunit of caspase-9 was found to interact with the BIR3 domain of XIAP (SEQ ID NO:3). Despite these advances, it was largely unclear how XIAP-mediated inhibition of caspase-9 actually occurs.

The targeted activation or inhibition of initiator caspases and compositions for effecting control of initiator caspase activity would be desirable. For example, in the treatment of cancers it would be desirable to promote selectively cell death by increasing apoptosis in tumor cells. This could have applications in the treatment of brain tumors such as neuroblastomas and glioblastomas, and in the treatment of refractory epilepsy.

Providing cells in need of increased apoptosis with a composition having polypeptide molecules with the surface groove of the BIR3 binding domain for recognition but lacking the four amino acids to inhibit initiator caspase-9 activity could be used to increase apoptosis in such cells. In, healthy tissues surrounding the tumor, inhibition of apoptosis could be used help protect the cells from the effects of cancer treatments. The selective delivery of apoptosis regulating agents may be used to achieve this effect.

Inhibition of apoptosis could be used to promote cell survival in neurons and consequently be useful therapies for neurodegenerative disorders, ischemic diseases, autoimmune diseases of the CNS, Parkinsonism, and to promote cell survival in sections of the spine. This may be achieved by providing cells in need of apoptosis inhibition with a composition including polypeptides having a BIR3 binding domain surface groove for recognition and the four amino acid residues for bonding to initiator caspases like caspase-9 in cells. Apoptosis in the cells can be suppressed by complexation of the caspase-9 with the polypeptide in a catalytically inactive form.

This invention relates, in one aspect, to a complex between a mammalian caspase-9 (SEQ ID NO: 1) and a polypeptide, including variants and pharmaceutically-acceptable salts thereof, the polypeptide including a BIR3 (SEQ ID NO:2) domain of an inhibitor of apoptosis protein (IAP). Preferably the BIR3 domain of the peptide is the BIR3 domain of XIAP (SEQ ID NO: 3) and includes any polypeptide characterized by having most of the amino acid sequence of BIR3 domain of XIAP (SEQ ID NO:3) that may yet be shortened on the N-terminal end, on the C-terminal end, or on both ends, by 1, 2, or a small number of residues and that nevertheless retains initiator caspase recognition, activity inhibiting binding, and a high binding affinity to processed caspase-9 and or Apaf-1 activated monomeric caspase-9 (apoptosome-activated caspase-9), (SEQ ID NO: 5). The polypeptide or its salts may be isolated and may include variants of the polypeptide that preferably have at least 80%, more preferably 85% or 90%, still more preferably 95%, 96%, 97%, 98%, or 99% identical to the BIR3 domain of XIAP (SEQ ID NO:3) such that the variant binds to the initiator caspase or an apoptosome of the initiator caspase and modifies and preferably inhibits its catalytic activity. A composition of the present invention includes a polypeptide having a BIR3 domain that forms a 1:1 complex or equivalently a heterodimer with an initiator caspase such as processed caspase-9 monomer (SEQ ID NO:1) or Apaf-1 activated monomeric caspase-9 (SEQ ID NO: 5). In one embodiment the polypeptide molecule in the composition includes amino acid residues for binding the polypeptide to the initiator caspase such that it inhibits the catalytic activity of the caspase. The composition may include pharmaceutically acceptable excipients. Preferably the complex prevents the caspase-9 activity from being expressed; in other words, the complex inhibits caspase-9 activity.

Another aspect of the invention includes an initiator caspase specific binding agent. The specific binding agent form a complex, and preferably a 1:1 complex or heterodimer, between an initiator caspase such as caspase-9 and or an Apaf-1 activated monomeric caspase-9 (apoptosome-activated caspase-9), (SEQ ID NO: 5) and the specific binding agent wherein the agent binds one or more of the residues on a caspase-9 molecule chosen from the group consisting of Leu 244, Pro247, Phe404, Phe406, Gln 245, Leu384, Leu385, Ala388, Cys403, Phe496, Ala316, Thr317, Pro318, Pro336, and Phe319. In preferred embodiments of the invention the specific binding agent binds two or more, three or more, four or more, or even more, of the above mentioned caspase-9 residues. The specific binding agent may be a peptidomimetic, polypeptide, or protein. The specific binding agent may include one or more residues chosen from the group consisting of a proline residue, a glycine residue, a leucine residue, and a histidine residue, which are disposed in space approximately as shown in FIG. 3. In one embodiment the initiator caspase specific binding agent includes a caspase-9 or apoptosome activated caspase-9 recognition binding sequence such as an XIAP-BIR3 domain, its variants or peptidomimetic equivalents thereof, and preferably also includes caspase-9 inhibiting amino acid residues functionally equivalent to Pro325, Gly326, His343, and Leu344 in BIR3 of XIAP or peptidomimetic equivalents thereof wherein the specific binding agent forms a heterodimer complex with an initiator caspase to inhibit its catalytic activity. In another embodiment the initiator caspase specific binding agent includes a caspase-9 or apoptosome activated caspase-9 recognition binding sequence such as an XIAP-BIR3 domain, its variants or peptidomimetic equivalents thereof, and includes point mutations, additions, or elimination of the caspase-9 inhibiting amino acid residues functionally equivalent to Pro325, Gly326, His343, and Leu344 in BIR3 of XIAP or peptidomimetic equivalents thereof, wherein the specific binding agent forms a heterodimer complex with an initiator caspase to modify its catalytic activity.

In another aspect of the invention, a method of forming a heterodimer 1:1 complex of caspase-9, an Apaf-1 activated monomeric caspase-9 (apoptosome-activated caspase-9), (SEQ ID NO: 5) or mixture thereof, with a composition having a specific binding agent that includes a BIR3 domain of XIAP or a peptidomimetic thereof is disclosed. The specific binding agent may include peptidomimetics, polypeptides, or proteins as well as their salts and or solvates. Preferably the specific binding agent also includes amino acid residues amino acid residues functionally equivalent to Pro325, Gly326, His343, and Leu344 in BIR3 of XIAP or their peptidomimetic equivalent. The method includes the step of contacting caspase-9, an Apaf-1 activated monomeric caspase-9 (apoptosome-activated caspase-9), (SEQ ID NO: 5) or mixture thereof with a composition that includes a BIR3 domain and amino acid residues functionally equivalent to Pro325, Gly326, His343, and Leu344 in BIR3 of XIAP or its peptidomimetic equivalent. In an important embodiment of the invention, the caspase-9 so contacted occurs within a cell, and in a further important embodiment the caspase-9 so contacted occurs within cells of a subject individual. Another embodiment of this aspect of the invention is a method of forming a heterodimer 1:1 complex of caspase-9 with a composition having purified and isolated form of an IAP such as XIAP or a composition having a purified and isolated form of XIAP with one or more point mutations at amino acid residues functionally equivalent to Pro325, Gly326, His343, and Leu344 in the BIR3 domain of XIAP.

In a further aspect of the invention, a method of inhibiting or modifying the activity of caspase-9 or its apoptosome is disclosed. The method include the step of contacting caspase-9, an Apaf-1 activated monomeric caspase-9 (apoptosome-activated caspase-9), (SEQ ID NO: 5), or a mixture thereof, with a composition having a specific binding agent that includes a surface groove of BIR3 and amino acid residues functionally equivalent to Pro325, Gly326, His343, and Leu344 in the BIR3 domain of XIAP in such a way that an activity modifying complex of caspase-9 or its apoptosome, and preferably a heterodimer complex, and the specific binding agent is formed. In another embodiment of the invention, the caspase-9 or the apoptosome caspase-9 activated complex activity so modified occurs within a cell, and in a further embodiment, the caspase-9 activity or the apoptosome caspase-9 activated complex occurs within cells of a subject individual. Another embodiment of this aspect of the invention is a method of inhibiting or modifying the activity of caspase-9 or the apoptosome caspase-9 activated complex by forming an complex, preferably a heterodimer, of caspase-9, the apoptosome caspase-9 activated complex, or a mixture thereof with a composition having purified and isolated form of XIAP or a composition having a purified and isolated form of XIAP with one or more point mutations at amino acid functionally equivalent to residues Pro325, Gly326, His343, and Leu344 in the BIR3 domain of XIAP.

An additional aspect of the invention relates to a method of treating a subject in need of inhibiting or modification of caspase-9 activity, the apoptosome caspase-9 activated complex activity, or a mixture of these, by steps that include administering a composition that includes a specific binding agent that may be a peptidomimetic, polypeptide, or protein. The specific binding agent including a BIR3 domain or peptidomimetic equivalent for initiator caspase recognition and amino acid residues functionally equivalent to Pro325, Gly326, His343, and Leu344 in the BIR3 domain of XIAP for inhibiting initiator caspase activity. The specific binding agent including a BIR3 domain or peptidomimetic equivalent for initiator caspase recognition and point mutations, addition. or elimination of amino acid residues functionally equivalent to Pro325, Gly326, His343, and Leu344 in the BIR3 domain of XIAP for modifying, for example by competitive binding, the activity of initiator caspases. Preferably the specific binding agent includes the BIR3 domain that is the BIR3 surface groove of XIAP. Another embodiment of the invention is a method of inhibiting or modifying the activity of caspase-9, the apoptosome caspase-9 activated complex, or a combination of these, by formation of an 1:1 complex of caspase-9 with a composition having a purified and isolated form of XIAP. Another embodiment of the invention is a method of inhibiting or modifying the activity of caspase-9 is by formation of a heterodimer 1:1 complex of caspase-9 with a composition having a purified and isolated form of XIAP with one or more point mutations at amino acid residues Pro325, Gly326, His343, and Leu344 in the BIR3 domain of XIAP.

Another embodiment of the present invention are isolated nucleic acid molecules comprising a nucleotide sequence encoding the amino acid sequence of caspase-9 ΔS, caspase-9 ΔL, or caspase-9 F404D. The invention is also directed to nucleic acid molecules comprising a nucleotide sequence complementary to the above-described sequences. Also provided for are nucleic acid molecules at least 80%, preferably 85% or 90%, still more preferably 95%, 96%, 97%, 98%, or 99% identical to any of the above-described nucleic acid molecules. Also provided for are nucleic acid molecules which hybridize under stringent conditions to any of the above-described nucleic acid molecules. The present invention also provides for recombinant vectors comprising these nucleic acid molecule, and host cells transformed with such vectors.

Also provided are isolated polypeptides comprising the amino acid sequence of caspase-9 ΔS, caspase-9 ΔL, or caspase-9 F404D. Also provided are polypeptides at least 80%, more preferably 85% or 90%, still more preferably 95%, 96%, 97%, 98%, or 99% identical to any of the above-described polypeptides. Also provided are methods for modifying apoptosis in a cell comprising contacting the cell with an above-described polypeptide.

### DESCRIPTION OF THE DRAWINGS

In part, other aspects, features, benefits and advantages of the embodiments of the present invention will be apparent with regard to the following description, appended claims and accompanying drawings where:

FIG. 1 Illustrates the crystal structure of caspase-9 in an inhibitory complex with XIAP-BIR3 (SEQ ID NO:6). (A) An overall view of the complex structure. XIAP-BIR3 binds to a large caspase-9 surface that is normally required for its catalytic activity. Caspase-9 is shown in blue, with the active site loops in purple and the N-terminus of the small subunit highlighted in gold. The catalytic residue, Cys287 on loop L2, is shown in ball and stick. The XIAP-BIR3 domain is colored green, with the bound zinc atom in red. (B) A perpendicular view (relative to panel A) of the caspase-9/BIR3 complex. (C) A schematic diagram of the published structure of the caspase-9 homo-dimer (SEQ ID NO:8) (Renatus et al., 2001). The active site loops of one of the two monomers (yellow) exist in active conformation while those of the other monomer (purple) are in an inactive conformation. (D) Superposition of the caspase-9/BIR3 complex (SEQ ID NO:6) with the caspase-9 homo-dimer. The coloring scheme is the same as in panels A-C. Note that XIAP-BIR3 (SEQ ID NO:3) completely overlap with one caspase-9 monomer. FIGS. 1, 2, and 3, were prepared using MOLSCRIPT (Kraulis, 1991) and GRASP (Nicholls et al, 1991).

FIG. 2 Illustrates the active site of the BIR3-bound caspase-9 (SEQ ID NO:6) exists in an unproductive conformation. (A) Superposition of the four active site loops from the BIR3-bound caspase-9 (blue) and the active (yellow) and inactive (purple) monomers of the caspase-9 homo-dimer. The active site confirmation of the BIR3-bound caspase-9 closely resembles that of the inactive caspase-9 monomer. (B) Surface representation of the active site loops in the BIR3-bound caspase-9. (C) Surface representation of the active site loops in the active caspase-9 monomer. Note the presence of the substrate-binding groove. (D) Surface representation of the active site loops in the inactive caspase-9 monomer.

FIG. 3 Illustrates the recognition of caspase-9 by the BIR3 domain of XIAP. (A) An overall view on the structure of the complex. Caspase-9 and BIR3 are shown as blue and green coil, respectively. A number of important amino acid interface residues from caspase-9 and BIR3 are colored yellow and purple, respectively. To illustrate the complementary binding, the transparent surface contour of caspase-9 is shown. (B) A stereo view on the interface centered around Pro325 and Gly326 of XIAP. The overall coloring scheme is the same as in FIG. 1. The side chains from key residues in caspase-9 and XIAP-BIR3 are colored yellow and gold, respectively. Hydrogen bonds are represented by red dashed lines. (C) A stereo view on the interface centered around His343 and Leu344 of XIAP. The side chain of His343 makes two hydrogen bonds to bridge caspase-9 and BIR3 whereas Leu344 packs against multiple hydrophobic residues in caspase-9. (D) A stereo depiction on the recognition of BIR3 by the N-terminal IAP-binding motif of caspase-9. The tetrapeptide motif of caspase-9 (Ala316-Thr317-Pro318-Phe319) (SEQ ID NO:9) binds to the conserved surface of BIR3. This binding is augmented by the close packing interactions from Pro336 and Pro338 of caspase-9. (E) Functional consequence of point mutations on the caspase-9 inhibiting amino acid residues of XIAP-BIR3. Cleavage of the procaspase-3 (SEQ ID NO:10) substrate by caspase-9 was performed in the absence or presence of various XIAP-BIR3 point mutants. The results were visualized by SDS-PAGE followed by Coomassie blue staining. The generation and purification of caspase-9 and XIAP-BIR3 mutant proteins and the caspase-9 assay are described in the Experimental Procedure. The procaspase-3 (C163A) precursor was used as the substrate.

FIG. 4 Illustrates that monomeric caspase-9 is inactive due to the lack of the supporting L2′ loop. (A) A schematic diagram of four caspase-9 variants. Using a co-expression strategy, these proteins were produced in their “cleaved” form (see Experimental Procedure for details). The approximate positions of the five loops in caspase-9 are indicated. (B) A time course of procaspase-3 cleavage by the four caspase-9 variants. p17 represents the cleaved product. Assays were performed as described in the Experimental Procedure.

FIG. 5 Is a schematic diagram of caspase-9 activation and inhibition. The full-length caspase-9 is colored green, with the prodomain (CARD) shown as a circle. The thickness of the black arrows indicates the preference of the equilibrium. Caspase-9 can be activated by the apoptosome comprising Apaf-1, cytochrome c, and the important co-factor dATP/ATP. Both isolated caspase-9 and the apoptosome-activated caspase-9 are subject to XIAP mediated inhibition.

### DETAILED DESCRIPTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

Apoptosis is essential for the development and homeostasis of metazoans. Alterations in apoptotic pathways have been linked to numerous human pathologies such as cancer and neuro-degenerative disorders. Apoptosis is executed by cascades of caspase activation. One of the well-documented cascades involves the initiator caspase, caspase-9, and the effector caspases, caspase-3 (SEQ ID NO: 11) and caspase-7 (SEQ ID NO:12). Many diseases include apoptotic cell death as part of the mechanism of pathology. Such mechanisms require the activity of caspase-9 (SEQ ID NO:1) as part of the caspase cascade leading to apoptosis. Examples of such pathologies may include Alzheimer's disease, stroke, arthritis, cachexia of AIDS, and still others.

Caspases are cysteine proteases that cleave their substrates after an aspartate or glutamate residue. Cell death or apoptosis occurs as a result of excessive cleavage of cellular machinery by the effector caspases. However, all effector caspases are produced in cells as a catalytically inactive zymogens and are proteolytically processed to become active proteases. This activation process strictly depends on the initiator caspases, which integrate the upstream apoptotic signals and initiate the caspase activation cascades. For example, active initiator caspase-9 (SEQ ID NO:1) cleaves and activates effector caspase-3 (SEQ ID NO:1) and caspase-7 (SEQ ID NO:12). Thus, the activation and inhibition of the initiator caspases constitute a central regulatory step in cellular physiology.

The crystal structure of caspase-9 (SEQ ID NO:1) in an inhibitory complex with the BIR3 domain of XIAP (SEQ ID NO:3), reveals a surprising mechanism of caspase inhibition. Through binding, the XIAP-BIR3 domain (SEQ ID NO:3) traps caspase-9 (SEQ ID NO:1) in a monomeric state and deprives it of any possibility of catalytic activity. A high binding affinity means that the dissociation constant for the complex is smaller than 1×10⁻⁶ M. Several lines of additional biochemical evidence to illustrate the mechanism of caspase-9 inhibition and regulation are provided.

For purposes of the present invention the term variants as used with respect to polypeptides preferably which are at least 80%, more preferably 85% or 90%, still more preferably 95%, 96%, 97%, 98%, or 99% identical to the BIR3 domain of XIAP and the variant binds to the initiator caspase or an apoptosome of the initiator caspase. For purposes of the present invention the term variants as used with respect to polynucleotides for preparing such polypeptides preferably refers to those polynucleotides which can be used to prepare polypeptides with at least 80%, more preferably 85% or 90%, still more preferably 95%, 96%, 97%, 98%, or 99% identical the BIR3 domain of XIAP and the polypeptide binds to the initiator caspase or an apoptosome of the initiator caspase.

Through crystallization and structure determination it was determined that the BIR3 domain of XIAP readily forms a tight complex with caspase-9, (SEQ ID NO:6), and inhibits its catalytic activity with a potency similar to that of the intact full-length XIAP (SEQ ID NO:13). X-ray crystallograpy is one method that could be used to determine the structure and binding sites of other specific binding agents with initiator caspases like caspase-9. The structure of caspase-9 with various polypeptides, peptidomimetics, their variants, and point mutants may be determined using the methods disclosed herein. In the present invention, the mechanism of XIAP-mediated inhibition of caspase-9, was determined through the crystal structure of a caspase-9/XIAP-BIR3 complex (SEQ ID NO:6). It was possible to generate crystals of the catalytic domain of caspase-9 (residues 139-416) in an inhibitory complex with the XIAP-BIR3 domain (residues 252-350). The crystals in this inhibitory complex are in the spacegroup P6,22 and diffract X-rays beyond 2.4 Å resolution (Table 1). The caspase-9 moiety in the asymmetric unit was located by Molecular Replacement using the atomic coordinates of the active half of the caspase-9 dimer as the initial search model (PDB code IJXQ). The electron density for the bound BIR3 domain became immediately apparent after preliminary refinement. The final atomic model of the inhibitory complex has been refined to a crystallographic R factor of 23.0% (R_{free}23.5%) at 2.4 Å resolution (Table 1).

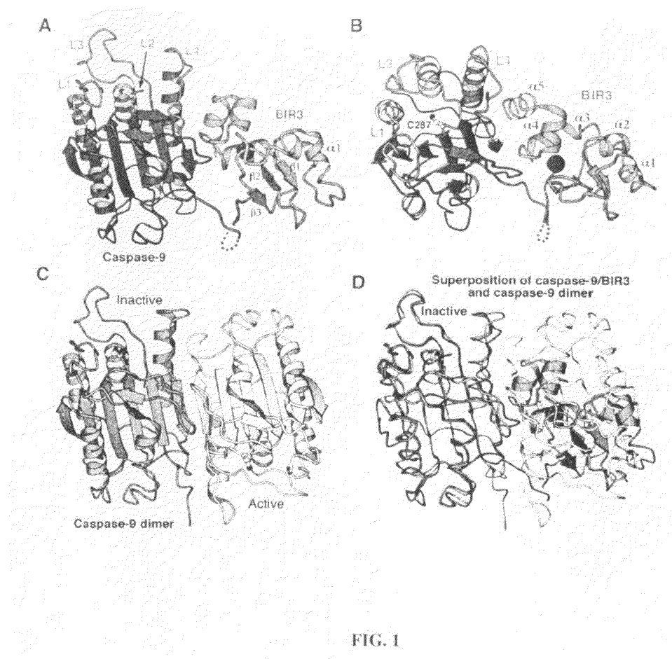

Overall the structure of the caspase-9/BIR3 complex shows that the XIAP-BIR3 domain forms a hetero-dimer with one caspase-9 monomer (FIGS. 1A & 1B). Caspases are thought to exist as homo-dimers. All 18 published caspases structures, including both the initiator caspases and the effector caspases, identify a homo-dimeric arrangement mediated by a predominantly hydrophobic interface (see Protein Data Bank, <URL http://www.rcsb.org). Recent studies indicate that, at least for caspase-3 and caspase-7, the formation of a homo-dimer is a prerequisite for any catalytic activity because one of the important supporting loops (L2′) for the active site of one monomer comes from the adjacent monomer. Thus, the BIR3 domain of XIAP appears to trap caspase-9 in a monomeric state, eliminating any possibility of forming a productive active site conformation.

In the complex, the XIAP-BIR3 domain forms a large continuous interface with the caspase-9 monomer, resulting in the burial of 2200 Å² exposed surface area (FIGS. 1A & 1B). On one side of the interface, helix α5 and the linker sequence between helices α3 and α4 of BIR3 pack closely against the hydrophobic surface of caspase-9. On the other side. the N-terminus of the small subunit of caspase-9 reaches out to interact with a conserved surface group on XIAP-BIR3 (FIGS. 1A and 1B).

XIAP-BIR3 traps caspase-9 in an inactive conformation. Previous structural studies on the dimeric caspase-9 reveal that the active site in one monomer exists in a productive conformation while the other active site is unraveled in the adjacent monomer (Renatus et al., 2001) (FIG. 1C). Interestingly, the structure of the BIR3-bound caspase-9 in the inhibitory complex is very similar to that of the inactive half of the caspase-9 dimer (FIG. 1D), with a root-mean-square deviation (rmsd) of 0.97 Å for all 221 Cα atoms. In particular, the active site loops of the BIR3-bound caspase-9 closely resemble those of the inactive half of the caspase-9 dimer (FIG. 1D).

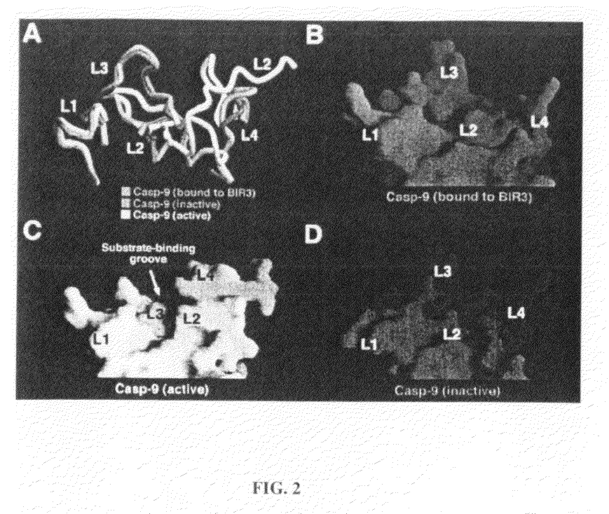

To examine this scenario in detail, a comparison of the four active site loops from the BIR3-bound caspase-9 with those from the active half as well as the inactive half of the caspase-9 homo-dimer (FIG. 2A) was made. All 48 Cα atoms of the active site loops can be superimposed with in rmsd of 1.3 Å between the BIR3-bound caspase-9 and the inactive half of caspase-9. For these two cases, the L1, L2, and L3 loops exhibit nearly identical conformations whereas the L4 loops are in the same general location (FIGS. 2A, 2B, and 2D). In this inactive confirmation, the substrate-binding groove is partially occupied by the L3 loop itself. In sharp contrast, there is a large difference between the active site conformations of the BIR3-bound caspase-9 and the active half of the caspase-9 homo-dimer (FIGS. 2A, 2B, and 2C), resulting in 5.7 Å rmsd for the same 48 aligned Cα atoms. Thus, the XIAP-BIR3 domain not only sequesters caspase-9 in a monomeric state but also traps the active site loops in their unproductive conformations.

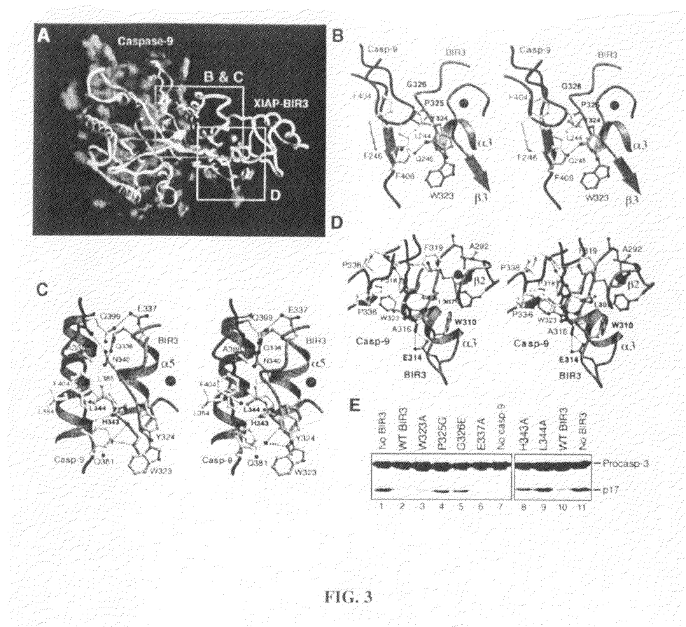

Recognition of caspase-9 by the XIAP-BIR3 domain involves a large protein-protein interface as well as a predicted interaction between the N-terminus of the caspase-9 small subunit and a highly conserved surface groove on BIR3. This recognition is dominated by a large collection of van der Waals contacts and further supported by 11 intermolecular hydrogen bonds at the interface (FIG. 3).

At the periphery of the protein-protein interface, two non-polar residues (Pro325 and Gly326) between helices α3 and α4 of BIR3 closely stack against a hydrophobic surface formed by Leu244, Pro247, Phe404, and Phe406 of caspase-9 (FIG. 3B). These interactions are supported by a specific hydrogen bond between Gln245 of caspase-9 and the backbone carbonyl group of Trp323. Interestingly, Leu244, Gln 245, and Pro247 all reside in a protruding loop that is unique to caspase-9. This characteristic loop, with a previously undefined function, is found to play an important role in binding the BIR3 domain of XIAP to caspase-9.

In the center of the protein-protein interface. Leu344 and His343 from BIR3 anchor the recognition of caspase-9 (FIG. 3C). Leu344 makes multiple van der Waals interactions to a hydrophobic pocket formed by four residues (Leu384, Leu385, Ala388, and Cys403) of caspase-9. His343 accepts an inter-molecular hydrogen bond from a caspase-9 backbone amide group while simultaneously making van der Waals contacts to Cys 403, Phe404, and Phe496 of caspase-9 (FIG. 3C).

The N-terminal four amino acids of the caspase-9 small subunit (Ala316-Thur3 17-Pro318-Phe319) conform to the Smac-like IAP-binding motif. This peptide sequence by itself is sufficient for the binding to XIAP-BIR3 and mutation of this sequence abolished BIR3-mediated inhibition of caspase-9 due to the loss of binding. This tetrapeptide (from caspase-9) was predicted to bind to the conserved surface groove of BIR3 in the same manner as the N-terminus of the mature Smac protein. Indeed, this interaction is just as predicted, with Ala316 playing the anchoring role in this part of the interface (FIG. 3D). Interestingly, this IAP-binding motif does not just bind to the BIR3 domain in isolation; it also packs against two adjacent caspase-9 residues, Pro336 and Pro338, through van der Waals contacts (FIG. 3D). These interactions mold the caspase-9 peptide-BIR3 binding into the larger and continuous protein-protein recognition interface (FIG. 3A).

Pro336 and its adjacent residues of caspase-9 constitute the core element of the L2′ loop in stabilizing the productive conformation of the active site loops in the structure of the caspase-9 homo-dimer (Renatus et al., 2001). However, in the inhibitory caspase-9/BIR3 complex, this region is involved in stabilizing the interactions between the IAP-binding motif of caspase-9 and the BIR3 domain. This analysis further reinforces the notion that XIAP-BIR3 not just sequesters caspase-9 in its monomeric form but also traps the active site loops in their unproductive conformations.

Mutational analysis was used to corroborate this structural observation, a caspase-9 assay was devise using its physiological substrate, procaspase-3 zymogen, and the ability of various XIAP-BIR3 point mutants to inhibit caspase-9 was investigated. Similar tests could be used to determine the activity of other specific binding agents such as polypeptides, peptidomimetics, their variants, and point mutants. A mutation on the catalytic residue, Cys163 to Ala, was introduced in the substrate procaspase-3 to prevent its self-activation or cleavage. As anticipated, the wild type (WT) caspase-9 cleaved the procaspase-3 precursor into p17 and p12 fragments (FIG. 3E, lane 1) and incubation with the WT BIR3 protein (residues 252-350) resulted in the efficient inhibition of the activity (lane 2). In contrast to the WT protein, mutation of any of the four caspase-9 activity inhibiting amino acid residues of BIR3 (P325G, G326E, H343A, and L344A) led to loss of this inhibition as judged by the cleavage of procaspase-3 precursor (FIG. 3E, lanes 4, 5, 8, and 9). The result that H343A can no longer inhibit caspase-9 confirms an earlier report. These residues make important contributions to the recognition and sequestration of the caspase-9 monomer (FIGS. 3B-3D); mutation of any of these residues presumably destabilizes the interface, allowing the caspase-9 restoration of its catalytic activity. It is of particular note that none of these mutations affects the conserved surface groove on BIR3; thus caspase-9 is still able to bind to the mutated BIR3 domain but is no longer subject to its inhibition.

These observations also confirm the important concept that recognition of caspase-9 by IAPs is necessary but not sufficient for its inhibition. Although the mutant XIAP-BIR3 forms a stable complex with caspase-9, it cannot effectively inhibit caspase-9 catalytic activity. Similarly, the BIR3 domain from either c-IAP1 or c-IAP2 can bind to the IAP-binding motif of caspase-9 (data not shown); yet neither c-IAP1 nor c-IAP2 is expected to inhibit caspase-9. These reasons are clear: Gly326 of XIAP is replaced by a charged and bulky residue Arg in c-IAP1 and c-IAP2. In addition, His343 and Leu344 or XIAP are replaced by Gln-Gly and Gln-Ala in c-IAP1 and c-IAP2, respectively. These changes are expected to disrupt the packing interactions of the protein-protein interface between caspase-9 and BIR3 and hence are unable to prevent the catalytic activity of caspase-9.

Amino acid residues in the polypeptides binding to the initiator caspases of the present invention may include naturally occurring amino acids and artificial amino acids. Incorporation of artificial amino acids such as beta or gamma amino acids and those containing non-natural side chains, and/or other similar monomers such as hydroxyacids are also contemplated, with the effect that the corresponding component is polypeptide-like in this respect and bind to the initiator caspase, preferably mammalian caspase-9, and either inhibit their catalytic activity or prevent inhibition of the initiator catalytic activity. “Proteins”, “peptides” and “poly peptides” are composed of a chain of amino acids connected one to the other by peptide bonds between the alpha-amino and carboxyl groups of adjacent amino acids.

A salt of the peptidomimetic, specific binding agent, or the polypeptide of the present invention includes salts with physiologically acceptable bases, e.g. alkali metals or acids such as organic or inorganic acids, and is preferably a physiologically acceptable acid addition salt. Examples of such salts are salts thereof with inorganic acids (e.g. hydrochloric acid, phosphoric acid, hydrobromic acid or sulfuric acid, etc.) and salts thereof with organic acids (e.g. acetic acid, formic acid, propionic acid, fumaric acid, maleic acid, succinic acid. tartaric acid, citric acid, malic acid, oxalic acid, benzoic acid, methanesulfonic acid or benzenesulfonic acid, etc.)

The peptidomimetic, specific binding agent, or the polypeptide of the present invention may include solvent molecules within their crystal lattice. Such hydrates, in the case of water molecules, or solvates in the ease of water molecules and or organic solvents such as but not limited to ethanol may have one or more water or solvent molecules present within the crystal lattice of the compounds.

The invention also provides for reduction of the subject initiator caspase activity modifying polypeptides to generate mimetics, e.g. peptide or non-peptide agents, which are able to mimic binding of the authentic polypeptides having the BIR3 binding groove for caspase-9 recognition, and the four caspase-9 activity inhibiting amino acids or point mutations of the four caspase-9 activity inhibiting amino acids. Such mutagenic techniques may be particularly useful for mapping the determinants of a polypeptide which participate in modifying the initiator caspase and IAP interactions involved in, for example, binding of the subject polypeptide with BIR3 binding domain to a caspase-9 polypeptide. To illustrate, the four caspase-9 activity inhibiting residues of a subject BIR3 and the surface groove of a subject BIR3 which are involved in molecular recognition of caspase-9 can be determined and used to generate BIR3-derived peptidomimetics which bind to caspase-9 and, like the authentic XIAP-BIR3, inhibit activation of the caspase-9. Similar methods may be used to generate peptidomimetics of binding but non-inhibiting polypeptide point mutants of a BIR3. By employing, for example, scanning mutagenesis to map the amino acid residues of a particular BIR3 polypeptide involved in binding a caspase-9 or apoptosome caspase-9 complex, peptidomimetic compounds (e.g. diazepine or isoquinoline derivatives) can be generated which mimic those residues in binding to the caspase-9 or apoptosome caspase-9 oligomer. For instance, non-hydrolyzable peptide analogs of such residues can be generated using benzodiazepine (e.g., see Freidinger et al. in Peptides: Chemistry and Biology, G. R. Marshall ed., ISCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), .beta.-turn dipeptide cores (Nagai et al. (1985) Tetrahedron Lett 26:647; and Sato et al. (1986) J Chem Soc Perkin Trans 1:1231), and .beta.-aminoalcohols (Gordon et al. (1985) Biochem Biophys Res Commun 126:419; and Dann et al. (1986) Biochem Biophys Res Commun 134:71).

The present invention incorporates U.S. Pat. Nos. 5,446,128, 5,422,426 and 5,440,013 in their entireties as references which disclose the synthesis of peptidomimetic compounds and methods related thereto. The compounds of the present invention may be synthesized using these methods. The present invention provides for peptidomimetic compounds which have substantially the same three-dimensional structure as those compounds described herein.

In similar fashion, identification of mutations in caspase-9 or apoptosome caspase-9 oligomer which effect binding to a XIAP-BIR3 polypeptide can be used to identify potential peptidyl fragments of caspase-9 or apoptosome caspase-9 oligomer which can competitively bind a XIAP-BIR3 polypeptide and interfere with its ability to inhibit the caspase. These and other peptidyl portions of caspase-9 or the apoptosome can be tested for binding to XIAP-BIR3 polypeptides or its variants using, for example, the procaspase-3 zymogen.

Another aspect of the invention pertains to an antibody specifically reactive with one of the subject XIAP-BIR3 proteins. For example, by using peptides based on the cDNA sequence of the subject XIAP-BIR3 protein, anti-XIAP-BIR3 antisera or anti-XIAP-BIR3 monoclonal antibodies can be made using standard methods. A mammal such as a mouse, a hamster or rabbit can be immunized with an immunogenic form of the peptide (e.g., an antigenic fragment which is capable of eliciting an antibody response). Techniques for conferring immunogenicity on a protein or peptide include conjugation to carriers or other techniques well known in the art. For instance, a peptidyl portion of the protein represented by SEQ ID No. 3 can be administered in the presence of adjuvant. The progress of immunization can be monitored by detection of antibody titers in plasma or serum. Standard ELISA or other immunoassays can be used with the immunogen as antigen to assess the levels of antibodies.

Following immunization. anti-XIAP-BIR3 antisera can be obtained and, if desired, polyclonal anti-XIAP-BIR3 antibodies isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, an include, for example, the hybridoma technique (originally developed by Kohler and Milstein, (1975) Nature, 256: 495-497). as the human B cell hybridoma technique (Kozbar et al., (1983) Immunology Today. 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the CCR-protein of interest and the monoclonal antibodies isolated.

The term antibody as used herein is intended to include fragments thereof which are also specifically reactive with a XIAP-BIR3 polypeptide or its variants. Antibodies can be fragmented using conventional techniques and the fragments screened for utility in the same manner as described above for whole antibodies. The antibody of the present invention is further intended to include bispecific and chimeric molecules.

Both monoclonal and polyclonal antibodies (Ab) directed against the subject XIAP-BIR3 polypeptides, and antibody fragments such as Fab′ and F(ab′)₂, can be used to block the action of particular XIAP-BIR3 and allow the study of the apoptosis.

Antibodies which are specifically immunoreactive with one or more IAP-BIR3 polypeptides of the present invention can also be used in immunohistochemical staining of tissue samples in order to evaluate the abundance and pattern of expression of the IAP-BIR3 polypeptide family, or particular members thereof. Anti-IAP-BIR3 antibodies can be used diagnostically in immuno-precipitation and immuno-blotting to detect and evaluate levels of one or more IAP-BIR3 polypeptides in tissue or cells isolated from a bodily fluid as part of a clinical testing procedure. For instance, such measurements can be useful in predictive valuations of the onset or progression of tumors. Likewise, the ability to monitor certain IAP-BIR3 levels in an individual can allow determination of the efficacy of a given treatment regimen for an individual afflicted with such a disorder. Diagnostic assays using anti-IAP-BIR3 antibodies, such as anti-XIAP-BIR3 antibodies, can include, for example, immunoassays designed to aid in early diagnosis of a neoplastic or hyperplastic disorder, e.g. the presence of cancerous cells in the sample.

One embodiment of the present invention are peptidomimetic compounds having the biological activity of XIAP-BIR3 for forming a heterodimer complex with a mammalian caspase-9 initiator caspase, wherein the compound has a bond, a peptide backbone or an amino acid component replaced with a suitable mimic. Examples of unnatural amino acids which may be suitable amino acid mimics include .beta.-alanine, L-.alpha.-amino butyric acid, L-.gamma.-amino butyric acid, L-.alpha.-amino isobutyric acid, L-.epsilon.-amino caproic acid, 7-amino heptanoic acid, L-aspartic acid, L-glutamic acid, cysteine (acetamindomethyl), N-.epsilon.-Boc-N-.alpha.-CBZ-L-lysine, N-.epsilon.-Boc-N-.alpha.-Fmoc-L-lysine, L-methionine sulfone, L-norleucine, L-norvaline, N-.alpha.-Boc-N-.delta.CBZ-L-ornithine, N-.delta.-Boc-N-.alpha.-CBZ-L-ornithine, Boc-p-nitro-L-phenylalanine, Boc-hydroxyproline, Boc-L-thioproline.

As used herein, the term “pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®. Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

When administered to a subject or patient, such polypeptides or specific binding agents of XIAP-BIR3 and variants thereof may be cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

Also provided by the invention arc pharmaceutical compositions comprising therapeutically effective amounts of polypeptide products of the invention, their salts, or peptidomimetics thereof together with suitable diluents, preservatives, solubilizers, emulsifiers, adjuvants and/or carriers. An “effective amount” as used herein refers to that amount which provides a therapeutic effect, such as initiation or inhibition of apoptosis for a given condition and administration regimen. Such compositions may be liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. The choice of compositions will depend on the physical and chemical properties of the polypeptide having the activity of an XIAP-BIR3 polypeptide. For example, a product which includes a controlled or sustained release composition may include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors.

Embodiments of the of the present invention such as peptidomimetics, polypeptides, specific binding agents, antibodies, nucleic acids and compositions including them may be in the forms such as solids, liquids, or as aerosols. These compositions may incorporate protective coatings, protease inhibitors or permeation enhancers for various routes of administration, including but not limited to parenteral, pulmonary, nasal, oral, injection or infusion by intravenous, intraperitoneal, intracerebral, intramuscular, intraocular, intraarterial or intralesional.

As noted above, pharmaceutical compositions also are provided by this invention. These compositions may contain any of the above described effectors, DNA molecules, vectors or host cells, along with a pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.

In addition, the pharmaceutical compositions of the present invention may be prepared for administration by a variety of different routes, including for example intraarticularly, intracranially, intradermally, intrahepatically, intramuscularly, intraocularly, intraperitoneally, intrathecally, intravenously, subcutaneously or even directly into a tumor. In addition, pharmaceutical compositions of the present invention may be placed within containers, along with packaging material which provides instructions regarding the use of such pharmaceutical compositions. Generally, such instructions will include a tangible expression describing the reagent concentration, as well as within certain embodiments, relative amounts of excipient ingredients or diluents (e.g., water, saline or PBS) which may be necessary to reconstitute the pharmaceutical composition. Pharmaceutical compositions are useful for both diagnostic or therapeutic purposes.

In addition to the compounds disclosed herein having naturally-occurring amino acids with peptide or unnatural linkages, the present invention also provides for other structurally similar compounds such as polypeptide analogs with unnatural amino acids in the compound. Such compounds may be readily synthesized on a peptide synthesizer available from vendors such as Applied Biosystems.

Polypeptides of the present invention include, but are not limited to, naturally purified products, chemically synthesized polypeptides, and polypeptides produced by recombinant techniques. Expression of polypeptides by recombinant techniques may result in different post-translational modifications, dependent on the host cell. These modified forms of the polypeptides are also encompassed by the claimed invention.

It would be readily recognized by one of skill in the art that some amino acid residues of XIAP-BIR3, c-IAP1, c-IAP-2, caspase-9ΔS, caspase-9ΔL, or caspase-9 F404D could be varied without significant effect on the structure or function of the protein. Such variations include deletions, insertions, inversions, repeats, and type substitutions. Guidance concerning which amino acid changes are likely to be phenotypically silent can be found in Bowie et al., Science 247:1306-1310 (1990).

The polypeptides of the present invention are 80%, more preferably 85% or 90%, still more preferably at least 95%, 96%, 97%, 98%, or 99% identical to the above-described polypeptides. Preferably, these IAP-BIR3 polypeptides, their variants, salts, and peptidomimetics thereof with modify caspase-9 activity. A skilled artisan is fully aware of possible amino acid substitution that arc less likely or not likely to significantly affect protein function.

The polypeptides of the invention may be used for the purpose of generating polyclonal or monoclonal antibodies using standard techniques known in the art (Klein, J., Immunology: The Science of Cell-Noncell Discrimination, John Wiley & Sons, N.Y. (1982); Kennett et al., Monoclonal Antibodies, Hybridoma: A New Dimension in Biological Analyses, Plenum Press, N.Y. (1980); Campbell. A., “Monoclonal Antibody Technology,” In: Laboratory Techniques in Biochemistry and Molecular Biology 13, Burdon et al. eds., Elseiver, Amsterdam (1984); Harlow and Lane, Antibodies, A Laboratory Manual, Cold Spring Harbor Laboratory, N.Y. (1988)). Such antibodies may be used in assays for determining gene expression and for screening expression libraries. Purified protein would serve as the standard in such assays.

The present inventors have shown that XIAP-BIR3 and its point mutations modify the caspase-9 induced apoptosis in cells. Thus, another embodiment of the present invention is a method of inducing programmed cell death in a cell comprising contacting the cell with a polypeptide or pepdiomimetic described above. For the purpose of controlling apoptosis in one or more cells, the polypeptides of the present invention can be administered to a cell in vitro or in vivo.

The polypeptides may be administered to the cell exogenously. The polypeptides may also be administered through recombinant expression. For example, homologous recombination can be used to express the polypeptides of the invention in cells. Extrachromosomal nucleic acids with the appropriate nucleotide sequence for XIAP-BIR3. c-IAP1, c-IAP2 and their variants can also be introduced into cells.

Induction of apoptosis can be used to treat malignant and pre-malignant conditions, and autoimmune disorders. Malignant and pre-malignant conditions may include solid tumors, B cell lymphomas, chronic lymphocytic leukemia, prostate hypertrophy, preneoplastic liver foci and resistance to chemotherapy.

Monomeric caspase-9 is catalytically inactive due to loss of the L2′ loop. Previous studies on effector caspases demonstrate that a productive conformation of the active site on one monomer involved the participation of the supporting L2′ loop on the adjacent monomer, which forms a loop bundle with the L2 and L4 loops through specific interactions. This result indicates that an effector caspases is in its dimeric form to exhibit any catalytic activity. Since the conformations of the active site loops are highly conserved among the effector and the initiator caspases, the L2′ loop is likely to be used for the initiator caspases as well. This hypothesis predicts that monomeric caspase-9 is catalytically inactive.

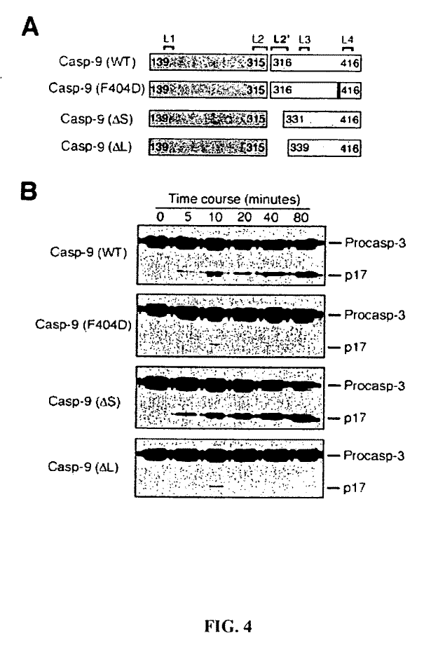

To examine this hypothesis, a monomeric caspase-9 was generated by mutating Phe404, which resides in the center of the homo-dimerization interface, to a negatively charged residue Asp (FIG. 4A). This mutation is expected to eliminate homo-dimerization of caspase-9 as burying two charged residues in the center of a predominantly hydrophobic interface is energetically extremely unfavorable. Indeed, this mutant caspase-9 (F404D) (SEQ ID NO: 25) exists exclusively as a monomer in solution (data not shown and see later). As anticipated, caspase-9 (F404D) did not exhibit any detectable enzymatic activity (FIG. 4B), despite the presence of all sequence elements required to form an active site.

Next, it was determined whether the L2′ loop in caspase-9 plays the same role as in caspases-3 and -7. Using a co-expression strategy, three caspase-9 variants (FIG. 4A) were generated, each of which contains an invariant large subunit (residues 139-315) and a distinct small subunit. Thus, these caspase-9 variants represent their “cleaved” or “active” form. The only difference is that, relative to the WT caspase-9, the ΔS (SEQ ID NO:23) and ΔL (SEQ ID NO: 24) variants contain deletion of residues 316-330 and 316-338, respectively (FIG. 4A). Removal of the fragment 316-330 does not affect any residue implicated in the stabilization of the active site conformation and hence should not have any negative impact on the catalytic activity of caspase-9. However, since the removal of residues 331-338 eliminates the formation of the loop-bundle, caspase-9 (ΔL) was expected to be inactive.

In subsequent in vitro caspase-9 assays, equal amounts of the caspase-9 variants were incubated with the procaspase-3 (C163A) substrate; the cleavage efficiency was monitored by SDS-PAGE and Coomassie staining (FIG. 4B). In complete agreement with the structure-based prediction, caspase-9 (ΔL) did not exhibit a detectable level of catalytic activity compared to the WT protein. In contrast, caspase-9 (ΔS) was approximately 2-fold more active than the WT protein (FIG. 4B). This is likely due to the elimination of the 15 flexible residues (315-330), which may impede substrate entry into the active site during catalysis. These modified inhibitor caspase-9 variants may be used in a gene therapy to modify apoptosis in cells.

These data demonstrate that the L2′ loop plays an indispensable role in stabilizing the conformation of the four active site loops (L1-L4) of caspase-9. This is the primary reason why a monomeric caspase-9 is inactive in solution. To further confirm this conclusion, Asp293 was mutated to Ala in caspase-9. Asp293, conserved among several caspases, is located on loop L2 and makes important contacts to residues on the L2′ loop. Thus this mutation is expected to disrupt the formation of the loop bundle involving loops L2′ and L4. Indeed, caspase-9 (D293A) exhibited an undetectable level of activity compared to the WT enzyme (data not shown).

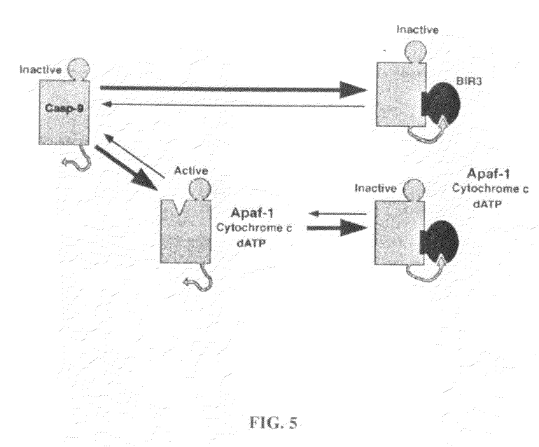

Without wishing to be bound by theory, a mechanistic paradigm on the regulation of caspase-9 activation and inhibition has emerged from these results (FIG. 5). At the basal state, both the procaspase-9 zymogen (SEQ ID NO:21) and the processed caspase-9 (SEQ ID NO:1) exist mostly as a monomer. These monomers have the potential to be activated by Apaf-1, for example, or inhibited (FIG. 5). XIAP may potently inhibits the catalytic activity of caspase-9 by using the BIR3 domain to hetero-dimerize with a caspase-9 monomer through the same interface that is required for the homo-dimerization of caspase-9 (FIG. 5). Thus, XIAP may trap caspase-9 in an inactive monomeric state, preventing any possibility of its catalytic activation (FIG. 5). Furthermore, the four active site loops from caspase-9 in the BIR3-bound caspase-9 exist in an unproductive conformation, and the fifth loop, loop L2′, is directly involved in the interaction between XIAP and caspase-9 (FIG. 3D). Thus the caspase-9/BIR3 structure also shows, in a broad sense, how a protein inhibitor can mess up the active state of a protease by trapping half of it (the monomer) in an inactive state. This mechanism prevents the assembly of a functional protease.

Caspase-9, one the best-characterized initiator caspases, plays an important role in apoptosis and directly activates the effector caspases-3 and 7. Although XIAP potently inhibits the catalytic activity of both caspase-9 and caspases-3 and -7. the underlying mechanisms are entirely different. In the case of the effector caspases, the active site is occupied by a small peptide sequence immediately preceding the BIR2 domain of XIAP (SEQ ID NO:19). Although unique in its own features, this mechanism falls into the frequently observed theme in the protease/inhibitor paradigm of inhibition by blocking the active site. For caspase-9, however, only the inactive monomer is trapped by the BIR3 domain of XIAP (SEQ ID NO:3) through an extensive protein-protein interface. Thus complete inhibition of enzymatic activity by XIAP is achieved without even touching the active site of caspase-9.

The recognition interface between caspase-9 and XIAP-BIR3 has two components. The binding between the IAP-binding tetrapeptide of caspase-9 and the conserved surface groove on XIAP-BIR3 (SEQ ID NO:22) is necessary but not sufficient for any XIAP-mediated inhibition. An additional protein-protein interface is present to direct the inhibition specificity. For example, despite the removal of a 15-residue peptide containing the Smac-like IAP-binding motif in the small subunit, the enzymatic activity of the resulting caspase-9 can still be inhibited by XIAP. In this case, although the N-terminus of the small subunit (AISS) alone is unable to form a stable complex with the BIR3 domain of XIAP, it can do so in the context of the caspase-9 protein, because the other significant protein-protein interface cooperates with this weak peptide-BIR3 binding to yield a stable complex.

Caspases were mainly regarded as a constitutive homo-dimers. This concept was derived from well over a dozen crystal structures, which showed again and again that both the initiator and the effector caspases are homo-dimers. However, careful evaluation of previous data really only reveals that the active effector caspases are homo-dimers. The reason why an effector caspase by itself can homo-dimerize in order to have any catalytic activity lies in the fact that the active site of a caspase monomer needs the support of an additional sequence element, the L2′ loop, which cannot be provided by the caspase monomer itself. Thus, dimerization can drive the activation of the initiator caspases, caspase-9. This concept is further supported by a report that both the processed caspase-9 (SEQ ID NO:1) and the procaspase-9 zymogen (SEQ ID NO:21) exist mostly as a monomer in solution (Table 2). This conclusion is supported using analytical ultra-centrifugation analysis, which represents the ideal method for the determination of molecular weights for macromolecular assemblies. The mechanism of Apaf-1-mediated activation of caspase-9 may have nothing to do with the dimerization process. The reason is that dimerization merely provides the L2′ loop for the active site of one monomer. If the apoptosome can somehow substitute for the badly needed L2′ loop for the caspase-9 monomer, it can certainly be activated without homo-dimerization (FIG. 5).

Various aspects of the present invention will be illustrated with reference to the following non-limiting examples.

### EXAMPLE 1

This example describes the preparation of proteins, polypeptide, and the preparation of caspase-9 variants of the present invention. All constructs were generated using a standard PCR-based cloning strategy, and the identities of individual clones were verified through double stranded plasmid sequencing. To minimize self-cleavage in bacteria, the catalytic subunit of caspase-9 (residues 139-416, in vector pET-21b) was co-expressed with the BIR3 domain of XIAP (residues 252-350, in vector pBB75) in Escherichia coli strain BL21(DE3), A serendipitous bonus from this co-expression is a large quantity of unprocessed procaspase-9 zymogen. The soluble fraction of the caspase-9/BIR3 complex and the procaspase-9 zymogen in the E. coli lysate were purified using a Ni-NTA (Qiagen) column, and further fractionated by anion-exchange (Source-15Q, Pharmacia) and gel-filtration chromatograph (Superdex-200, Pharmacia). Recombiant active caspases-7 and missense mutant of caspase-9 and XIAP-BIR3 were over-expressed and purified as described (Chai et al., 2001a; Chai et al., 2001b). For the three caspase-9 deletion variants (FIG. 4A), the large and the small subunits were co-expressed and purified as described (Chai et al., 2001b).

### EXAMPLE 2

This example describes the structure of inhibiting heterodimer complexes of the present invention. Crystallization and data collection. Crystals of the caspase-9/BIR3 complex were grown by the hanging-drop vapor diffusion method by mixing protein with an equal volume of reservoir solution. The well buffer contains 100 mM Tris, pH 8.0, 1.0 M potassium monohydrogen phosphate, and 0.2 M sodium chloride. Small crystals appeared after three weeks, with a typical size of 0.1×0.1×0.3 mm³. The crystals belong to the space group P6₅22, contain one complex in each asymmetric unit, and have a unit cell dimension of a=b=104.42 Å and c=170.31 Å. Crystals were equilibrated in a cryoprotectant buffer containing well buffering plus 24% glycerol, and were flash frozen in a −170° C. nitrogen stream. The native data were collected at the CHESS beamline A1. The data were processed using the software Denzo and Scalepack (Otwinowski and Minor, 1997).

Structure determination and refinement. The structure was determined by Molecular Replacement, using the software AMoRe (Navaza, 1994). The atomic coordinates of the active half of the caspase-9 dimer (PDB code IJXQ) were used for rotational and subsequent translational searches against a 15-3.0 Å data set, which yielded a single promising solution with high correlation factors. The candidate solution was checked in the program “O” (Jones ct al., 1991) and subjected to rigid body refinement using CNS (Terwilliger and Berendzen, 1996). The electron density for the BIR3 domain was unambiguous. The BIR3 moiety was built in and the caspase-9/BIR3 complex was refined further by simulated annealing using CNS. The final refined atomic model (R_{free}˜0.235) contains residues 256-346 for XIAP-BIR3, residues 140-288, 316-320, and 333-416 for caspase-9, 215 ordered water molecules, and one zinc atom at 2.4 Å resolution.

### EXAMPLE 3

This example illustrates the construction of a caspase-9 assay. The reaction was performed at 37° C. under the following buffer conditions: 25 mM HEPES. pH 7.5, 100 mM KCl, and 1 mM dithiothreitol (DTT). The substrate (procaspase-3, C163A) concentration was approximately 80 μM. Caspase-9 variants were diluted to the same concentration (0.3 μM) with the assay buffer. Reactions were stopped with the addition of equi-volume 2× SDS loading buffer and boiled for three minutes. The samples were applied to SDS-PAGE and the results were visualized by Coomassie-staining.

### EXAMPLE 4

This example describes the use of analytical ultracetrifugation for measuring the molecular weight of various proteins and polypeptides and its use for determining the presence or absence of inhibitor caspase-9 homo-dimers in solution.

To accurately determine the basal state of caspase-9 in solution, the molecular weight of caspase-9 was examined by sedimentation equilibrium analysis using analytical ultra-centrifugation (Table 2). Little, if any, variation in molecular weight as a function of rotor speed was observed for any of the caspase-9 samples, indicating that the protein behaves mostly as a single species in solution (data not shown). Both the processed caspase-9 and the unprocessed procaspase-9 zymogen were found to have a molecular weight consistent with that of a monomer. In addition, this analysis confirms that the XIAP-BIR3 domain forms a stable hetero-dimer with the caspase-9 monomer (Table 2). In contrast, this method demonstrates that the active caspases-7, which is known to be dimeric, indeed exhibits a molecular weight consistent with that of a dimmer (Table 2).

Analytical ultracentrifugation. Protein samples were prepared in 10 mM Tris-HCl, pH 8.0, 100 mM NaCl, and 2 mm DTT. All sedimentation equilibrium experiments were carried out at 4° C. using a Beckman Optima XL-A analytical ultracentrifuge equipped with an An60 Ti rotor and using six-channel, 12 mm path length, charcoal-filled Epon centerpieces and quartz windows. Data were collected at four rotor speeds (10,000, 15,000, 20,000, and 25,000 rpm) and represent the average of twenty scans using a scan step-size of 0.001 cm. Partial specific volumes and solution density were calculate using the Sednterp program. Data were analyzed using the WinNONOLIN program from the Analytical Ultracentrifugation Facility at the University of Connecticut (Storrs, Conn.). The results show that caspase-9 exists mostly as a monomer in solution and a single species of caspase-9 has been observed in solution by gel filtration as well as by analytical ultra-centrifugation.

TABLE 1
Data collection and statistics from the crystallographic analysis

| Beamline | CHESS-A1 |
|---|---|
| Spacegroup | P6₅22 |
| Resolution (Å) | 99.0-2.3 Å |
| Total observations | 415,375 |
| Unique observations | 23,136 |
| Data coverage (outer shell) | 99.7% (100%) |
| R_{sym} (outer shell) | 0.071 (0.525) |
| Refinement: | |
| Resolution range (Å) | 20.0-2.4 Å |
| Number of reflections (all) | 22104 |
| Data coverage | 100% |
| R_{working}/R_{free} | 0.230/0.235 |
| Number of atoms | 2806 |
| Number of waters | 215 |
| R.m.s.d. bond length (Å) | 0.012 |
| R.m.s.d. bond angles (degree) | 2.09 |
| Ramachandran Plot: | |
| Most favored (%) | 84.6 |
| Additionally allowed (%) | 14.3 |
| Generously allowed (%) | 1.1 |
| Disallowed (%) | 0.0 |

R_{sym}=ΣₕΣᵢ|I_{h,i}−Iₕ|/ΣₕΣᵢI_{h,i}, where Iₕ is the mean intensity of the i observations of symmetry related reflections of h. R=Σ|F_{obs}−F_{calc}|/ΣF_{obs}, where F_{obs}=Fₚ, and F_{calc} is the calculated protein structure factor from the atomic model (R_{free} was calculated with 5% of the reflections). R.m.s.d. in bond lengths and angles are the deviations from ideal values, and the r.m.s.d. deviation in β factors is calculated between bonded atoms.

TABLE 2
A summary of the analytical ultracentrifugation measurements.

| | | Molecular Weight (Dalton) | |
|---|---|---|---|
| Sample | Concentration | Observed | Calculated |
| Caspase-9 (active) | 20 μM | 28,500 ± 700 | 31,297 |
| | 10 μM | 31,120 = 1,540 | 31,297 |
| Caspase-9/XIAP-BIR3 | 20 μM | 39,380 ± 1,220 | 42,973 |
| | 10 μM | 41,060 ± 1,530 | 42,973 |
| | 5 μM | 42,200 ± 2,440 | 42,973 |
| Caspase-7 | 20 μM | 54,530 ± 1,070 | 29,865 |
| | 10 μM | 49,720 ± 1,070 | 29,865 |
| Procaspase-9 zymogen | 20 μM | 29,920 ± 1,400 | 31,457 |
| | 10 μM | 27,840 ± 2,150 | 31,457 |

Molecular weight represents global analysis of data collected at four rotor speeds 10K, 15K, 20K, 25K rpm. All data were collected at 4° C. The Caspase-9/XIAP-BIR3 sample contains the wild-type caspase-9 residues 139-315 and 316-416 and XIAP residues 252-350. The active caspase-9 contains residues 139-315 and 316-416 except that residues Glu304-Asp305-Glu306 have been replaced by three Ala residues to reduce limited proteolysis by the intrinsic enzymatic activity of caspase-9. The procaspase-9 zymogen contains residues 139-416. The active caspase-7 contains residues 51-198 and 200-303.

Although the present invention has been described in considerable detail with reference to certain preferred embodiments thereof, other versions arc possible. Therefore the spirit and scope of the appended claims should not be limited to the description and the preferred versions contain within this specification.

What is claimed is:

1. A modified IAP comprising a portion of a naturally occurring IAP wherein said modified IAP comprises at least one BIR domain having:
a proline in a 3-dimensional position corresponding to P325 of BIR3 of XIAP;
a glycine in a 3-dimensional position corresponding to G326 of BIR3 of XIAP;
a histidine in a 3-dimensional position corresponding to H343 of BIR3 of XIAP; and
a leucine in a 3-dimensional position corresponding to L344 of BIR3 of XIAP.

2. The binding agent of claim 1, wherein said portion of a naturally occurring IAP is a portion of c-IAP1.

3. The binding agent of claim 1, wherein said portion of a naturally occurring IAP is a portion of c-IAP2.

4. The binding agent of claim 1, wherein said portion of a naturally occurring IAP is a portion of XIAP.

5. The binding agent of claim 1, wherein said portion of a naturally occurring IAP is a portion of ML-IAP.

6. The binding agent of claim 1, wherein said modified IAP further comprises one or more BIR1, one or more BIR2, one or more BIR3 or a combination thereof.

7. The binding agent of claim 1, wherein said modified IAP forms a complex with a caspase.

8. The binding agent of claim 1, wherein said modified IAP forms a complex with caspase-9.

9. The binding agent of claim 1, wherein said modified IAP binds to a caspase with a higher affinity than a naturally occurring IAP.

10. A caspase binding compound prepared by the method comprising:
applying a three-dimensional molecular modeling algorithm to the atomic coordinates of at least a portion of an IAP bound to a caspase;
electronically screening stored spatial coordinates of candidate compounds against the spatial coordinates of the at least a portion of the IAP;
identifying a compound that is substantially similar to the at least a portion of the IAP; and
synthesizing the identified compound.

11. The compound of claim 10, wherein the method for preparing the compound further comprises identifying one or more amino acids necessary for IAP-initiator caspase binding.

12. The compound of claim 10, wherein the caspase is caspase-9.

13. The compound of claim 10, wherein the IAP is XIAP.

14. The compound of claim 10, wherein the compound forms a 1:1 complex with the caspase.

15. The compound of claim 10, wherein the compound comprises:
a proline in a 3-dimensional position corresponding to P325 of BIR3 of XIAP;
a glycine in a 3-dimensional position corresponding to G326 of BIR3 of XIAP;
a histidine in a 3-dimensional position corresponding to H343 of BIR3 of XIAP; and
a leucine in a 3-dimensional position corresponding to L344 of BIR3 of XIAP.

16. A method for preparing a caspase inhibitor comprising:
applying a three-dimensional molecular modeling algorithm to atomic coordinates of at least a portion of an IAP bound to a caspase;
electronically screening stored spatial coordinates of candidate compounds against the spatial coordinates of the at least a portion of the IAP;
identifying a compound that is substantially similar to the at least a portion of the IAP; and
synthesizing the identified compound.

17. The method of claim 16, further comprising identifying one or more amino acids necessary for IAP-initiator caspase binding.

18. The method of claim 16, wherein the caspase is caspase-9.

19. The method of claim 16, wherein the IAP is XIAP.

20. The method of claim 16, wherein the at least a portion of the IAP comprises:
a proline in a 3-dimensional position corresponding to P325 of BIR3 of XIAP;
a glycine in a 3-dimensional position corresponding to G326 of BIR3 of XIAP;
a histidine in a 3-dimensional position corresponding to H343 of BIR3 of XIAP; and
a leucine in a 3-dimensional position corresponding to L344 of BIR3 of XIAP.

21. A method for identifying a caspase inhibitor comprising:
applying a three-dimensional molecular modeling algorithm to atomic coordinates of at least a portion of an IAP bound to a caspase;
electronically screening stored spatial coordinates of candidate compounds against the spatial coordinates of the at least a portion of the IAP;
identifying a compound that is substantially similar to the at least a portion of the IAP; and
displaying the identified compound.

22. The method of claim 21, further comprising identifying one or more amino acids necessary for IAP-initiator caspase binding.

23. The method of claim 21, wherein the caspase is caspase-9.

24. The method of claim 21, wherein the IAP is XIAP.

25. The method of claim 21, wherein the at least a portion of the IAP comprises:
a proline in a 3-dimensional position corresponding to P325 of BIR3 of XIAP;
a glycine in a 3-dimensional position corresponding to G326 of BIR3 of XIAP;
a histidine in a 3-dimensional position corresponding to H343 of BIR3 of XIAP; and
a leucine in a 3-dimensional position corresponding to L344 of BIR3 of XIAP.

26. The method of claim 21, further comprising synthesizing the identified compound.

27. An IAP binding compound prepared by the method comprising:
applying a three-dimensional molecular modeling algorithm to atomic coordinates of at least a portion of XIAP, wherein said at least a portion at least comprises P325, G326, H343, and L344;
electronically screening stored spatial coordinates of candidate compounds against the spatial coordinates of the at least a portion of XIAP;
identifying a compound that is substantially complementary to the at least a portion of XIAP; and
synthesizing the identified compound.

28. The compound of claim 27, wherein the compound forms a 1:1 complex with an IAP.

29. The compound of claim 27, wherein the compound forms a 1:1 complex with XIAP.

30. The compound of claim 27, wherein the compound releases IAP mediated inhibition of a caspase.

31. The compound of claim 27, wherein the compound releases IAP mediated inhibition of caspase-9.

32. The compound of claim 27, further comprising one or more mimetic.
